(19) United States
(12) Patent Application Publication
(10) Pub. No.: US 2010/0041999 A1
(43) Pub. Date: Feb. 18, 2010

(54) Process for quantitative display of blood flow

(76) Inventors: Thomas Schuhrke, Munich (DE); Guenter Meckes, Munich (DE); Joachim Steffen, Westhausen (DE); Hans-Joachim Miesner, Aalen (DE); Frank Rudolph, Aalen (DE); Werner Nahm, Buehlerzell (DE)

Correspondence Address: ECKERT SEAMANS CHERIN & MELLOTT, LLC, U.S. STEEL TOWER, 600 GRANT STREET, PITTSBURGH, PA, 15219-2788, US

(21) Appl. No.: 12/462,037

(22) Filed: Jul. 28, 2009

(30) Foreign Application Priority Data
Jul. 28, 2008 (DE) 10 2008 040 803.4

(51) Int. Cl. A61B 6/00; G06K 9/00

(52) U.S. Cl. 600/476; 382/134

(57) ABSTRACT

A method for the quantitative representation of the blood flow in a tissue or vascular region based on the signal of a contrast agent injected into the blood. In the process, several individual images of the signal emitted by the tissue or vascular region are recorded at successive points in time and are stored. For image areas of stored individual images the respective point in time is determined at which the signal has exceeded a certain threshold value and this point in time is represented for each of the image areas.

### BACKGROUND OF THE INVENTION

The invention relates to a quantitative method for the representation (display) of the blood flow in a patient.

Several methods for observing and determining the blood flow in tissue and vascular regions are known in which a chromophore such as indocyanine green, for example, is applied. The fluorescent dye can be observed as it spreads in the tissue or along the blood vessels using a video camera. Depending on the area of application, the observation can be non-invasive or in the course of surgery, for example via the camera of a surgical microscope.

Many methods are known, where only the relative distribution of the fluorescent dye in the tissue or in the blood vessels is examined qualitatively in order to draw conclusions concerning their blood flow. For example, conclusions are made about the blood flow and diagnoses are provided by watching an IR video recorded during surgery. It is also known to record an increase in the in brightness of the fluorescence signal over time at all or at selected image points and in this manner create a time chart of the signal emitted by the fluorescent dye. The profile of the recorded formation plot provides the physician with information about potential vascular constrictions or other problems in this area of this image point. One example for this is provided in DE 101 20 980 A1. However, the method described in the DE 101 20 980 A1 goes beyond the qualitative analysis and embarks on a path towards a quantitative determination of the blood flow at every image point.

The objective forming the basis of the invention is to provide medical professionals with additional aids from which they can draw conclusions concerning blood flow problems and that can support making a diagnosis.

This objective, as well as other objectives which will become apparent from the discussion that follows, are achieved, according to the present invention, by the method and apparatus described below.

According to the invention, the contrast agent flowing into the tissue or vascular area is observed by recording the signal emitted by said contrast agent as a video, by splitting the video into individual images and storing the same, or by storing individual images directly, and by determining for several corresponding image areas, in particular image points of the individual images, the respective point in time at which the recorded image reaches a signal strength that is above a specified threshold value, in order to generate a two-dimensional representation of the respective inflow times, an offset representation relative to a starting time like, for example, the earliest found inflow time or the starting time of the recordings. The result is then a representation of inflow times assigned to the respective recorded image areas or image points, respectively. In an ideal case, the respective image areas of the individual images can be the same local image point or image area, that is, for example, if the resolution is reduced and image points are to be combined, a small number of adjacent image points, if different individual images have been recorded with the same resolution of exactly the same detail of the object, or according to the invention in one advantageous embodiment can also be corresponding image points or image areas in different individual images that still are to be assigned to each other, because the recording conditions have changed between the recordings, for example, object and shooting direction have moved in relation to each other or the resolution has been changed or the like. This will be explained in greater detail in a later section. Preferably, the injected contrast agent is a fluorescent dye, such as indocyanine green, for example. However, other dyes known for perfusion diagnostics can be used as well. The excitation of the fluorescence for generating the signal to be obtained occurs typically via a near infrared light source. An infrared camera, which is often a CCD camera or a CMOS camera and which can be an autonomous medical device or can be integrated in a surgical microscope, is used for recording. The generation of the individual images of the signals that are to be recorded occur either by splitting a video into individual images or directly through storing recorded individual images in certain time sequences. The individual images may be stored as a bitmap, for example. The time the threshold at the image point to be viewed is exceeded relative to a reference point in time constitutes the time offset after which the contrast agent in the blood has arrived at a location of the tissue or vascular region. This allows for a conclusion to be drawn about the flow behavior of the blood in the region. For the individual providing treatment this representation provides a valuable aid allowing recognition of flow blockages or constrictions. It is, therefore, a very important new diagnosis aid. The point in time when the threshold is exceeded can be derived in various manners. For example from the signal strength of the recorded signal itself, from the slope of the signal or by observing signal properties that are typical for the signal before and after the threshold value is exceeded.

Advantageously, the threshold value is defined below 25% of the maximum signal strength, and its preferred value is at 20% of the maximum signal strength. For values in this range, it can be expected that the noise level of the recording or of other background signals are not interpreted as the signal of the contrast agent while significant vessels with contrast agent flowing through them are captured If a lower threshold level were set, it would be possible to interpret the noise erroneously as the inflow time of the contrast agent, and if it were set too high, areas with a lesser blood flow, i.e., where the signal remains significantly below the maximum would not be captured. However, these areas might be the ones of greatest medical interest.

In one advantageous embodiment of the invention, the time offset is transferred into a color on a color scale such that a false color image is created based on which the flow behavior of the blood is visible. A false color image provides a very quick and intuitive overview of the time successions.

Preferably, the false color scale is selected such that an intuitive correlation to known anatomical terms exists. For example, the arterial character is emphasized by representing early points in time in red, while the venous character of other areas is emphasized by representing later points in time in blue. In this manner, the false color image is adjusted directly to a common manner of thinking of the individual providing treatment, and thus provides them with a very intuitive direct overview.

In an additional preferred embodiment a grayscale is selected as the scale for the points in time when the signal strength exceeds a certain threshold. This scale may have a slightly poorer resolution than a false color image, however, it is suited for the black-and-white representation.

In one additional preferred embodiment, prior to determining the point in time of exceeding the threshold value, a movement compensation is applied to the individual images. This means, the individual images are, if they are offset from each other, first placed on top of each other such that indeed the respective associated image points can be compared when determining the points in time. The underlying problem here is that the recording unit or the object to be recorded may move during recording. In such a case, the recorded images of the signals will be, at least slightly, shifted in relation to each other, such that this shift must first be reversed if one plans to receive a steady signal progression for each image point of the recorded object. Such a steady signal progression is the prerequisite for determining in a spatially resolved manner the time when the threshold value of the signal is exceed. Thus, without movement compensation, the points in time could be assigned falsely to the image points and could lead to an erroneous representation of the time offset. Preferably, the movement is compensated using edge detection, where edge images of the individual images are generated that can then be correlated in order to determine from it the shift vector. As soon as the shift vector of an individual image is determined, this individual image is shifted in relation to the previous image according to the shift vector. In one embodiment, the edge images of successive individual images are used for the correlation of the edge images. Preferably, however, the edge image of an individual image is correlated to a reference image that is generated by joining together the previous edge images of the individual images that have already been correlated to each other. In the course of this process, this creates a reference image that includes all the edges that have occurred in the individual images that have been correlated before. Any individual image can be used as the starting reference image, or an image where the total signal strength has exceeded a certain value or where it is determined in another fashion that the recorded signal has exceeded a noise level and is indeed the signal of the inflowing contrast agent. Generating the summed up reference image for the movement compensation is essential because individual images that are recorded at very different times can show a totally different edge structure because the signal may have already flattened in one area when it reaches the maximum in another area. It would then not be possible to properly correlate these very different images that have been recorded at different points in time.

In another advantageous embodiment, a brightness correction is applied to the individual images that takes into account changes in the recording conditions that affect the brightness of the signal. For example, the amplification factor at the camera can be adjusted such that a greater contrast range of the signal can be captured during recording. The intensity of the light source or other recording conditions can be adjusted as well such that the brightness correction may need to take several different parameters into account. For this purpose, changes in the recording conditions are stored together with the individual images, and during the brightness correction, the recorded signal values are converted to a common value range taking into account these stored data. This ensures that a steady signal progression occurs at every image point.

For a full understanding of the present invention, reference should now be made to the following detailed description of the preferred embodiments of the invention as illustrated in the accompanying drawings.

### BRIEF DESCRIPTION OF THE DRAWINGS

FIG. 1 shows a schematic sequence of a method for presenting the blood flow.

FIG. 2 shows an example of a profile of a brightness plot at one image point.

FIGS. 3a and b show examples of blood vessel representations without and with movement compensation.

FIGS. 4a and b show examples of time offset representations of false color representations converted to grayscale and as a grayscale image.

FIG. 5 shows schematically a surgical microscope for carrying out the method according to the invention.

### DESCRIPTION OF THE PREFERRED EMBODIMENTS

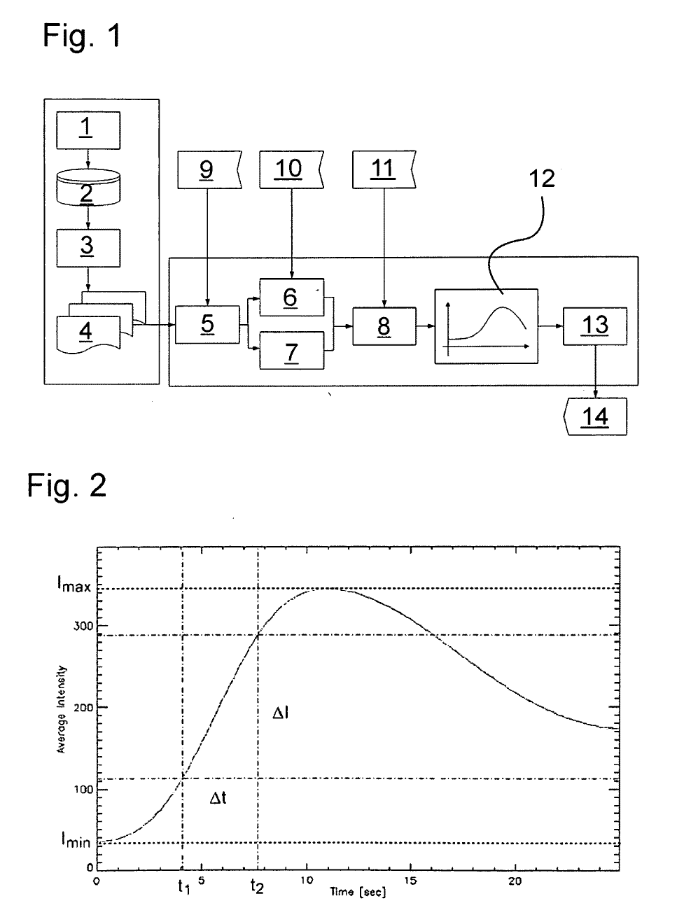

The preferred embodiments of the present invention will now be described with reference to FIGS. 1-5 of the drawings. Identical elements in the various figures are designated with the same reference numerals.

The complete system with the data flows and the individual processing steps is described in FIG. 1 and is used for presenting and evaluating the blood flow. The data are recorded using a video camera 1 in the infrared range, which is arranged at the surgical microscope—not shown—or is a component thereof. The recorded infrared videos are stored in a data memory 2 and are split into individual images 4 using a video player 3. Alternatively, it is also possible to store the images of the video camera 1 directly as individual images 4. A frequency of five frames 4 per second proved to be useful. They are then corrected in a single image correction step 5. In the process, the corrections for the edge drop, the dark offset or of non-linearities of the video camera 1 are carried out taking into account the required correction data 9. The data of the corrected individual images 4 are then stored in the form of compressed binary data (e.g., Motion JPEG2000 Data (MJ2)) or in the form of non-compressed binary data (e.g., bitmap). In the case of non-compressed binary data, access times are shorter and the evaluation is faster.

For the evaluation, the individual images 4 are transferred to the algorithms for the brightness correction 6 and movement correction 7. For the brightness correction 6, for example, the different amplification factors that have been set at the video camera 1 are taken into account during recording in order to adapt the video camera 1 to the different fluorescence strength of the tissue or vascular area to be recorded. They are documented during the recording as well, are stored on the data memory 2 as metadata 10 assigned to the video data and are computed with the individual images 4. During the movement correction 7, the positions of the recorded individual images 4 are aligned. The video camera 1 or the object, i.e., the tissue or vascular area to be recorded may move during video recording. In such cases, the individual images 4 are offset from each other. Thus, the individual images 4 must be re-aligned in order to evaluate the details visible in the individual images 4 without faults. This is exacerbated by the constantly changing image information in the individual images 4. To have an initial image for comparison purposes, a reference image is selected from among the individual images 4. The first image on which clear structures can be recognized can serve as an initial reference image. Using an edge detection method, all additional individual images 4 that are to be computed with the reference image are continuously examined for their degree of offset in comparison to the reference image. This offset is taken into account in all additional steps where several individual images 4 are involved. In particular the reference image is continuously updated by integrating the edge image of the following individual image that is offset to the correct position into the reference image.

The brightness determination 8 can be carried out following the corrections 6 and 7. For this purpose, first the position of the measurement range is determined in a measurement range determination 11. The measurement range for which the time offset representation has to be generated can be defined in a measurement range determination 11 via a measurement window or as a selection of specified measurement points. For example, a range of the recording can be selected if only this range is to have a time offset representation, or if the time offset representation is to be generated for a portion of the image points only in order to save computing time. The result of the brightness determination 8 is a brightness plot 12 as a function of the time as can be seen in FIG. 2. This brightness plot 12 is computed for all or at least for a sufficiently large sample of image points.

In an evaluation 13, numerous other representations 14, comprising individual results as well, can be supplied from these brightness plots 12 and the individual images 4. They can then be presented on the screen together with the individual images 4.

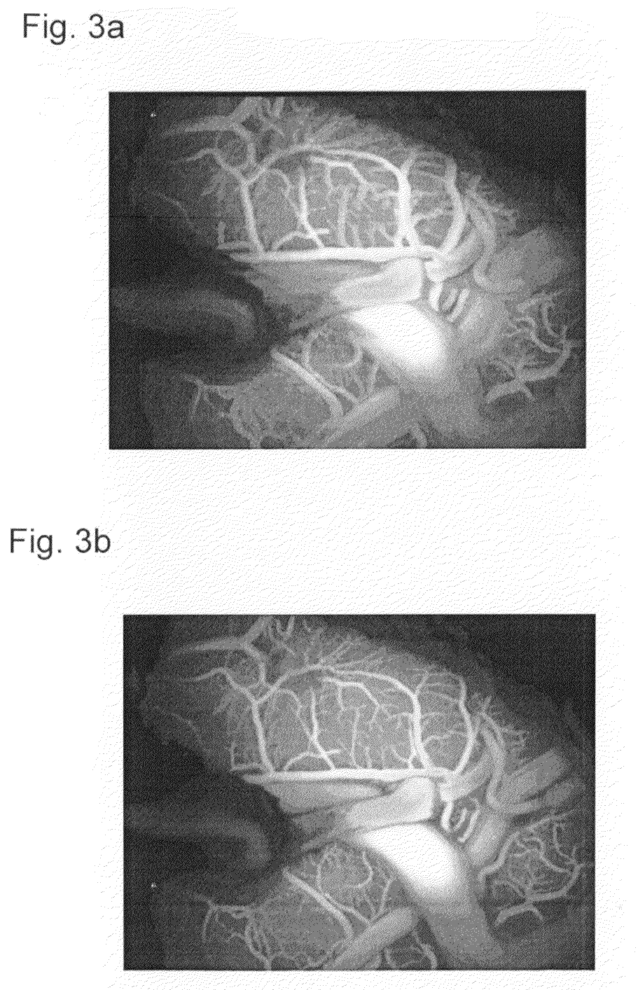

One example for this is a so-called blood vessel representation, where all vessels and all tissues through which fluorescence agents flowed appear light. This representation is generated by presenting the difference between the maximum and minimum brightness value for each image point of the superimposed individual images 4. With this maximum brightness for each image point, one obtains a relative, quantitative quantity for the blood flow at all positions. This enables the physician to recognize defects. Examples for blood vessel representations can be seen in FIGS. 3a and 3b. FIG. 3a shows a blood vessel representation that has been generated without movement compensation 7, while FIG. 3b shows an example with movement compensation 7. Clearly recognizable is the significantly better sharpness of the contours in FIG. 3b with movement compensation.

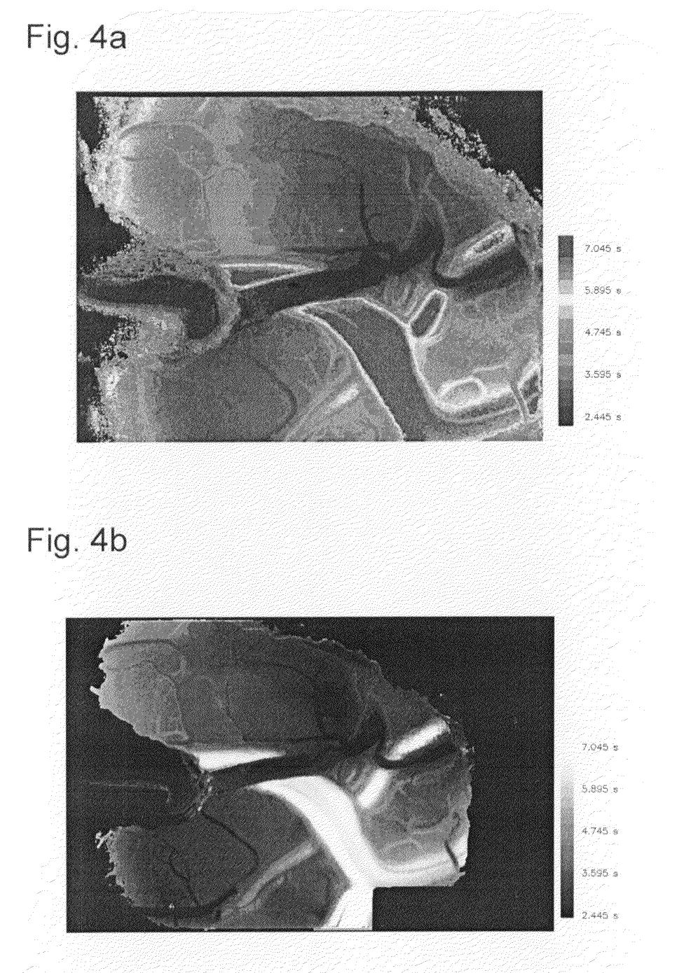

A two-dimensional false color image representing the time offset is provided for an additional representation 14. It can be seen in FIGS. 4a and 4b. FIG. 4a shows the onset time of the blood flow in a color representation converted to grayscale, whereby the bars on the right side show the false color scale, that is, the relationship between the selected colors and the respective elapsed time. The false color scale is selected such that an intuitive correlation to known anatomic terms exists. Accordingly, red is selected for an earlier point in time in order to emphasize the arterial character and blue for a later point in time to accent the venous character. In FIG. 4a, the color scale thus transitions from red (here at about 2.5 sec) to green (here at about 5 sec) and finally to blue (here at about 7 sec). In this manner, the physician receives a quick overview of the time when the blood arrived at which position of the blood vessel or of the tissue. Thus, using the time offset, information about the inflow and outflow of the blood in the blood vessels or in the tissue is made transparent. Because the conversion of the false color image into grayscale does not permit an unambiguous assignment of the colors, a similar representation 14 of a time offset in place of a false color image has been implemented as a grayscale image with a grayscale for black and white representations as are necessary here, for example, or also for black-and-white screens. This can be seen in FIG. 4b. Here, blood vessels into which the blood with the fluorescent dye flows immediately are shown dark while the blood vessels that the blood reaches later are shown very light. However, the grayscale representation has less information contents compared to the false color representation. Other types of representation such as a three-dimension representation, for example, where the third dimension is the time, are conceivable as well.

To generate the representation 14, a brightness plot 12 is computed for each image point based on all individual images 4 of the video. Then the point in time t₁ at which the brightness plot 12 has exceeded a certain threshold value I(t₁) is determined for each image point. The threshold value is defined as I(t₁)=Iₘᵢₙ+0.2×(Iₘₐₓ−Iₘᵢₙ). This point in time is converted to the respective color, grayscale or height and entered into the time offset representation, Iₘₐₓ and Iₘᵢₙ must be determined by comparing the recorded data of several individual images 4 in order to determine the threshold value I(t₁). To obtain a spatially resolved signal, it is extremely important to carry out a movement compensation first. Without movement compensation 7, the brightness plot 12 is not steady such that several Iₘₐₓ and Iₘᵢₙ could arise in each brightness plot 12. The same applies to the brightness correction 6. Without a brightness correction 6, a steady plot would also not arise for recording devices where the recording conditions may change during the recording of the individual images 4 and where the changes affect the brightness of the recorded individual images 4. Changes in the recording conditions may be necessary, for example, whenever a greater contrast range is to be covered.

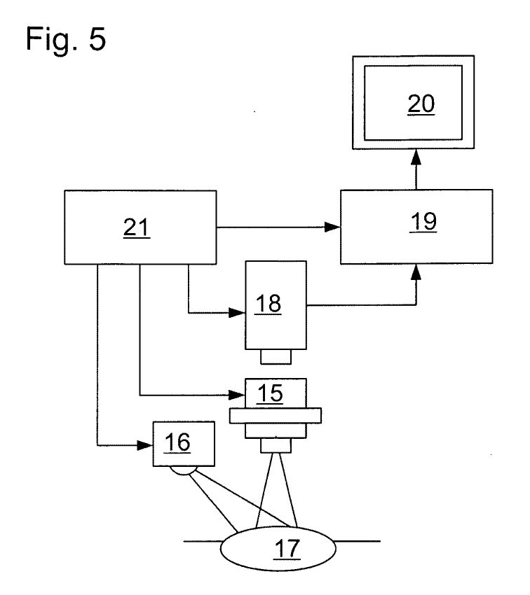

FIG. 5 shows schematically the essential components of a surgical microscope that can be used to apply the method according to the invention. The optics 15 of a surgical microscope reproduces an object 17, for example the head of a patient that is to be treated during surgery and is illuminated by a light source 16 of the surgical microscope in a camera 18. The camera 18 can also be a component of the surgical microscope. The image data recorded by the camera 18 are transferred to a computer unit 19 where they are evaluated. Medical quantities derived at the evaluation are then represented on the screen 20, potentially together with the recorded image. Similar to the computer unit 19, the screen 20 can be a component of the central surgical control but can also be a component of the surgical microscope. A control unit 21 controls the brightness of the light source 16 as well as the magnification factor and the aperture of the optics 15 and the amplification factor of the camera 18. In addition, the control unit 21 generates metadata that provide information about changes in the recording conditions that occur as soon as the control unit 21 adjusts a quantity that is to be controlled. These metadata are transferred from the control unit 21 to the computer unit 19, where they are assigned to the image data that have been provided to the computer unit 19 by the camera 18. Metadata and image data are stored, at least temporarily, by the computer unit 19 and are evaluated according to the method according to the invention. During the evaluation, the metadata are included with the image data. The results of the evaluation according to the invention are then displayed on the display unit 20, possibly together with the image data.

There has thus been shown and described a novel method and apparatus for quantitative display of blood flow which fulfills all the objects and advantages sought therefor. Many changes, modifications, variations and other uses and applications of the subject invention will, however, become apparent to those skilled in the art after considering this specification and the accompanying drawings which disclose the preferred embodiments thereof. All such changes, modifications, variations and other uses and applications which do not depart from the spirit and scope of the invention are deemed to be covered by the invention, which is to be limited only by the claims which follow.

What is claimed is:

1. A method for the quantitative representation of the blood flow in a tissue or vascular region based on the signal of a contrast agent injected into the blood, said method comprising the steps of:
recording and storing at successive points in time in an image sequence, several individual images of the signal emitted by the tissue or vascular region,
for shown areas of tissue or vascular regions determining the respective point in time at which the signal in the image sequence exceeds a certain threshold value, and
representing this point in time for the respective shown areas.

2. A method for the quantitative representation of the blood flow in a tissue or vascular region as set forth in claim 1, wherein the threshold value is less than 25% of the maximum of the achieved signal strength.

3. A method for the quantitative representation of the blood flow in a tissue or vascular region as set forth in claim 1, wherein the threshold value is at 20% of the maximum of the achieved signal strength.

4. A method for the quantitative representation of the blood flow in a tissue or vascular region as set forth in claim 1, wherein a brightness plot of the signal is obtained for each of the image areas to be viewed in order to determine the threshold value.

5. A method for the quantitative representation of the blood flow in a tissue or vascular region as set forth in claim 1, wherein the threshold value is defined in reference to the maximum signal intensity.

6. A method for the quantitative representation of the blood flow in a tissue or vascular region as set forth in claim 1, wherein the points in time for the image points are represented in the form of a false color image.

7. A method for the quantitative representation of the blood flow in a tissue or vascular region as set forth in claim 6, wherein early points in time are represented in red and later points in time in blue.

8. A method for the quantitative representation of the blood flow in a tissue or vascular region as set forth in claim 1, wherein the points in time for the image areas are represented in the form of a grayscale image.

9. A method for the quantitative representation of the blood flow in a tissue or vascular region as set forth in claim 1, wherein a movement compensation is applied for the individual images prior to the determination of the points in time.

10. A method for the quantitative representation of the blood flow in a tissue or vascular region as set forth in claim 9, wherein edge images of individual images are generated for the movement compensation using an edge detection method.

11. A method for the quantitative representation of the blood flow in a tissue or vascular region as set forth in claim 10, wherein edge images are correlated to each other in order to determine a shift factor.

12. A method for the quantitative representation of the blood flow in a tissue or vascular region as set forth in claim 11, wherein each correlation of the edge image of an individual image is carried out using a reference image that is developed by supplementing the edge images of two correlated and shifted individual images.

13. A method for the quantitative representation of the blood flow in a tissue or vascular region as set forth in claim 1, wherein a brightness correction is applied to the individual images prior to the determination of the points in time.

14. A method for the quantitative representation of the blood flow in a tissue or vascular region as set forth in claim 13, wherein metadata are recorded and stored for the brightness correction during recording of the individual images.

15. A surgical microscope for recording a fluorescence radiation of a contrast agent comprising a camera for recording an image sequence of an object and optics for reproducing the object in the camera, whereby the camera is connected to a computer unit for deriving medical quantities from an image sequence of medical image data or individual images of the image sequence, the improvement wherein the computer unit operates in accordance with a program for carrying out the method as set forth in claim 1.

16. An analysis system of a surgical microscope for recording a fluorescence radiation of a contrast agent, comprising a computer unit that operates in accordance with a program for performing the method as set forth in claim 1.
